(19) United States
(12) Patent Application Publication
(10) Pub. No.: US 2026/0069156 A1
(43) Pub. Date: Mar. 12, 2026

(54) Estimating Pulse Transit Time from Synchronized Sensor Signals of a Wearable Device

(71) Applicant: Samsung Electronics Co., Ltd., Suwon-si (KR)

(72) Inventors: Li Zhu, Saratoga, CA (US); Mehrab Bin Morshed, Gilroy, CA (US); Md Mahbubur Rahman, San Jose, CA (US); Weinan Wang, Piscataway, NJ (US); Jilong Kuang, San Jose, CA (US); Sharanya Arcot Desai, San Jose, CA (US)

(21) Appl. No.: 19/313,434

(22) Filed: Aug. 28, 2025

Related U.S. Application Data
(60) Provisional application No. 63/692,553, filed on Sep. 9, 2024.
(60) Provisional application No. 63/755,931, filed on Feb. 7, 2025.

(51) Int. Cl. A61B 5/024; A61B 5/00; A61B 5/1455; G06F 1/16

(52) U.S. Cl. CPC A61B 5/02416; A61B 5/1455; A61B 5/6803; A61B 5/7289; G06F 1/163

(57) ABSTRACT

In one embodiment, a method includes acquiring a motion signal by an IMU sensor of a wearable device and acquiring a PPG signal by a PPG sensor of the wearable device, where the PPG sensor is synchronized with the IMU sensor. The method further includes determining, from the IMU signal, an AVO of a wearer of the wearable device; determining from the PPG signal, a pulse arrival time of the wearer; and estimating, based on (1) the AVO determined from the IMU signals and (2) the pulse arrival time determined from the PPG signal, a PTT of the wearer.

### PRIORITY CLAIM

This application claims the benefit under 35 U.S.C. § 119 of U.S. Provisional Patent Application No. 63/692,553 filed Sep. 9, 2024 and U.S. Provisional Patent Application No. 63/755,931 filed Feb. 7, 2025, both of which are incorporated by reference herein.

### TECHNICAL FIELD

This application generally relates to estimating pulse transit time from synchronized sensor signals of a wearable device.

### BACKGROUND

Cardiovascular diseases are serious and common health conditions that affect millions of people worldwide. Cardiovascular diseases are a leading causes of global morbidity and mortality, and can have a pronounced impact on quality of life even when not fatal.

There are several metrics that are used to estimate or determine cardiovascular health. For example, blood pressure is a marker for many cardiovascular diseases, and detecting a person's blood pressure can be critical for early detection, effective management, and prevention of cardiovascular complications, such as stroke, heart failure, and coronary artery disease.

Pulse transit time (PTT), which measures the time it takes for a pulse wave to travel from the heart to a particular location on the body, is another marker for cardiovascular health. For example, blood pressure is inversely related to PTT, as the higher the central blood pressure, the higher the pulse wave velocity, and in turn, PTT becomes shorter. As another example, attributes of the vasculature system such as blood vessel stiffness are related to PTT.

### BRIEF DESCRIPTION OF THE DRAWINGS

FIG. 1 illustrates an example method for accurately and non-invasively detecting PPT signals from an IMU sensor and a PPG sensor of a wearable device.

FIG. 2. Illustrates an example implementation of an AVO detection block, among other things.

FIG. 3 illustrates an alternative example of a B-point detection algorithm that uses both IMU signals and PPG signals.

FIG. 4 illustrates an example computing system.

FIG. 5 illustrates an example system implementing the example method of FIG. 1.

### DESCRIPTION OF EXAMPLE EMBODIMENTS

Non-invasive monitoring of markers for cardiovascular health can improve the detection of cardiovascular conditions, both at a point in time and over time. However, many non-invasive techniques required a specialized, dedicated device to detect a cardiovascular-related marker, and often require supervision or application by a health professional, such as a nurse or doctor.

For example, traditional cuff-based blood pressure (BP) monitors operate intermittently, causing user discomfort and providing no real-time data, and often result in measurement inaccuracies due to cuff position errors, observer variability, and the “white coat effect,” where blood pressure readings are artificially elevated due to anxiety during clinical measurements.

Pulse Transit Time (PTT) measures the time it takes for a pulse wave to travel from the heart to a particular region of the body, and is related to a variety of cardiovascular markers and conditions. Conventionally, an ECG is used to determine PTT by identifying the R-wave (a particular cardiovascular component in an ECG signal) as the starting point of the cardiac cycle, then measuring the time it takes for the pulse wave to reach a peripheral location as detected by photoplethysmography (PPG), which uses emission and reflection of optical signals to detect pulse waves in the vasculature. However, major limitations prevent the success of this approach. For instance, the time between the R-wave and the actual aortic valve opening (pre-ejection period, or PEP) is not accounted for in the calculation, affecting the accuracy of PTT measurement. In addition, PEP is modulated by non-BP related factors, such as stress and gender, and therefore is variable both from person to person and for an individual over time, and so cannot simply be accounted for by using a fixed delay. Finally, the range of PEP is around 100 milliseconds, which contributes an unacceptable confounding factor for estimating PTT using this approach.

Alternatively, PTT is sometimes measured using two PPG sensors placed at different sites on the body, and then calculating the time difference between the peaks of the PPG waveforms detected at each site. However, this approach also has several limitations. First, as an optical sensor, PPG cannot measure exact aortic valve opening. PPG sensors are also very sensitive to their exact placement conditions, and precise placement is crucial for consistent PTT measurement, as slight variations in sensor position can dramatically affect PPG results. Body temperature, vascular compliance, and body positions can also influence the PTT measured by two PPG sensors. Second, acquiring and accurately placing two PPG devices presents a high barrier for adopting and using this approach. Third, wireless synchronization across different measurement sites remains presents a problem, as lack of synchronization prevents accurate PTT calculation even if the pulse waves are measured accurately at their respective PPG sites.

Accurate detection of aortic valve opening (proximal pulse timing) is important for accurately measuring PPT because any delay or error directly impacts the accuracy of PTT measurement. As discussed above, existing techniques do not address this problem, as ECG-based techniques introduce a PEP confounding factor that greatly reduces accuracy, and optical methods such as dual PPG are unable to detect precise aortic valve opening because they (1) measure peripheral pulse waves rather than the central cardiac event and (2) lack precise synchronization between the sensors worn at different body locations, thereby introducing errors to a PPT estimate.

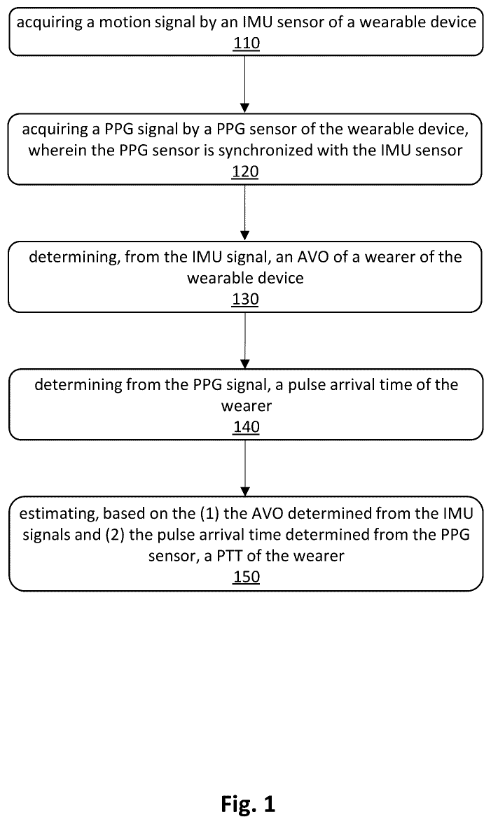

In contrast, the techniques described herein accurately and non-invasively estimate PPT signals using synchronized IMU and PPG signals from an IMU sensor and PPG sensor in a wearable device, such as an earbud. FIG. 1 illustrates an example method for accurately and non-invasively detecting PPT signals from an IMU sensor and a PPG sensor of a wearable device. Step 110 of the example method of FIG. 1 includes acquiring a motion signal by an inertial measurement unit (IMU) sensor of a wearable device. In particular embodiments, the IMU sensor may be a 6-axis IMU, which includes a 3-axis accelerometer and a 3-axis gyroscope.

In particular embodiments, the wearable device may be an earbud or a pair earbuds. In other embodiments, a wearable device may be a watch, a ring, etc. In embodiments in which the wearable device is an earbud, one or both of the IMU sensor and the PPG sensor described below may be located in the earbud such that when a person wears the earbud, then those sensors are on or near the concha, which is where people tend to wear earbuds, and has rich vascularization that can provide strong PPG signals.

Mechanical events within the heart, such as the opening of the aortic valve, correspond with specific points in the cardiac cycles (e.g., as illustrated by a Wiggens diagram) and these mechanical events can be accurately measured by an IMU sensor, as described herein. For example, a lumped-parameter model may be used to formalize the ballistocardiograph (BCG) force acting on the blood, which represents the ballistic (mechanical) forces created by the heart pumping blood through the vascular system.

Aortic valve opening (AVO) marks the onset of systolic blood ejection from the left ventricle into the aorta. This process generates subtle vibrations transmitted through thoracic cavity, neck, and cranial bones to the ear concha. The anatomical structure of ear concha makes it a natural amplifier for internal vibration, which makes an ear-worn device such as an earbud a good candidate for IMU-based detection of AVO.

Step 120 of the example method of FIG. 1 includes acquiring a PPG signal by a PPG sensor of the wearable device, where the PPG sensor is synchronized with the IMU sensor. This synchronization between IMU and PPG sensors in the wearable device is an important part of accurately detecting PPT signals using the techniques described herein.

Synchronization can be challenging because IMU and PPG sensors inherently operate using different sampling and digitization processes. In addition, the analog-to-digital conversion for PPG signals and the digital interface latencies for IMU signals tend to introduce timing discrepancies between these two sensors.

Particular embodiments of this disclosure therefore use a master clock of a controller (e.g., a microcontroller (MCU)) of a main processing unit that processes the sensor signals from each sensing modality. As described below, particular embodiments implement interrupt-driven sampling to ensure minimum time offset, and apply a timestamping mechanism for each sensor data point within firmware.

The high precision internal synchronization is achieved by a common MCU clock that triggers signal acquisition from each sensor. The IMU and PPG sensors share a common MCU that simultaneously triggers data acquisition for both sensors. Specifically, the MCU simultaneously triggers an analog front end for acquiring PPG signals and the IMU to acquire data points precisely at a variable but relatively high sampling rate, e.g., a sampling rate of 450 samples/second (equivalent to a sampling interval of about 2.2 ms). As PPT is extremely sensitive to timing errors between proximal timing events (aortic valve opening, detected by IMU) and distal timing events (pulse arrival, detected by PPG), even minor synchronization errors (˜2 milliseconds) can introduce blood pressure estimation inaccuracies of approximately 1 mmHg. High precision (sub-millisecond synchronization) ensures that (1) the IMU can reliably capture the mechanical event of AVO precisely at the true physiological onset of ventricular ejection and (2) that the PPG sensor precisely captures the distal pulse wave arrival, and that these events are each captured and timestamped according to the same clock. The precise synchronization between the IMU and PPG sensor minimizes temporal jitter, preserving physiological integrity in the PTT measurements. For example, particular embodiments may have a total synchronization error of less than 100 μs, due to an ADC conversion (from the PPG) having a delay of 10-50 μs, a delay due to the digital interface (the inter-integrated circuit) of about 5-20 μs, and a delay due to the MCU clock and interrupts of less than 1 μs.

In addition, the noise floor of an IMU is typically set relatively high, e.g., at 200 μG/√ Hz or even higher. This is because IMUs are typically used to detect a large range of motion, and a higher noise floor results in a higher detection range. However, the aortic valve opening generates extremely subtle mechanical vibrations (typically in the order of tens to hundreds of micro-g's) that propagate through body tissues to peripheral locations such as the ear, and the noise floor typically associated with IMUs makes such subtle signals undetectable. Therefore, particular embodiment tune the accelerometer noise floor of the IMU used in a wearable to, e.g., 70 μG/√ Hz, which significantly enhances sensitivity and detection capability for these subtle physiological signals. Utilizing such a low noise level ensures the accurate identification of the precise onset timing of AVO events, directly improving downstream PTT measurement accuracy.

In particular embodiments, a printed circuit board (PCB) in the wearable device that contains the IMU sensor is mounted in the wearable device so that, when worn, an IMU axis (e.g., the y axis) is aligned with the wearer's head-to-foot direction so as to maximize detection of heartbeat-induced vibrations in the user's body.

In particular embodiments, a wearable device may operate in two distinct modes related to cardiovascular data collection. For example, one mode may be an energy preservation mode, in which only the IMU (and only the accelerometer, in particular embodiment) is active. In addition, the accelerometer in the energy-preservation mode may sample at a much lower rate than in the PTT-sensing mode, e.g., at a rate of 25 samples per second, and with a much higher detection range (e.g., +/−16 g). In particular embodiments, the energy preservation mode may correspond to typical IMU sensing capabilities and therefore can be used to detect typical motion signals, allowing the wearable device to use a single IMU to perform the techniques of this disclosure (in the high-precision, PPT sensing mode) while also performing other motion-related functionality (in the energy preservation mode).

In the energy preservation mode, the accelerometer may detect motion signals. These motion signals are then analyzed (e.g., by the wearable device's processing unit, or by a connected device, e.g., smartphone, etc.) to determine whether the user is stationary. The user may be determined to be stationary when the user's motion (or motion over a period of time) is less than a threshold, such as, e.g., 1.5 g for an axis perpendicular to the ground and 0.5 g on the other two orthogonal axes. If the user is not stationary, then the energy-preservation mode continues. If the user is stationary, then the high-precision, PTT sensing mode may be enabled. In this mode, both the full IMU (e.g., both accelerometer and gyroscope) are active, as is the PPG sensor. And as described above, the sensors in this mode sample at a much higher rate (e.g., 450 samples per second) and the accelerometer operates at a higher precision with a lower noise floor (e.g., operates at +/−2 g with the relatively low noise floor described above). The wearable device may continue in the high-precision PTT mode until the user is no longer stationary, until a certain amount of time passes, until a number of PTT estimates are made, etc.

In particular embodiment, an amount of time in a high-precision mode may vary based on a risk profile of the wearer. For instance, for low-risk users (e.g., as determined by the user's physician, or on current/previous cardiovascular diseases, or on the user's CHA2DS-VASc score), the amount of time in the high precision mode may be limited to, e.g., a few times a day. In contrast, for high-risk users, the amount of time the wearable device spends in high-precision PTT mode may be increased. In particular embodiment, if a high-risk wearer is not stationary enough to enter high-precision mode, then the user may be sent a notification (e.g., via a sound from the earbud, via a notification displayed on a client device such as a smartphone, etc.) instructing the user to stay stationary so that the wearable device can enter the high-precision mode. In particular embodiment, the sensing frequency can be configured to occur periodically across difference sleep stages of a wearer.

In particular embodiments, a wearable device may include other sensors, such as one or more of a temperature sensor (e.g., a thermal sensor) for sensing body temperature or a microphone. For example, a microphone in the ear canal can capture heartbeat signals via mechanical vibration at relatively low frequencies, which can serve as a temporal signal that can be used to enhance detection of cardiovascular events. As another example, a thermal sensor may be used to detect the wearer's body temperature, which can be used as an input to estimate blood pressure of the wearer.

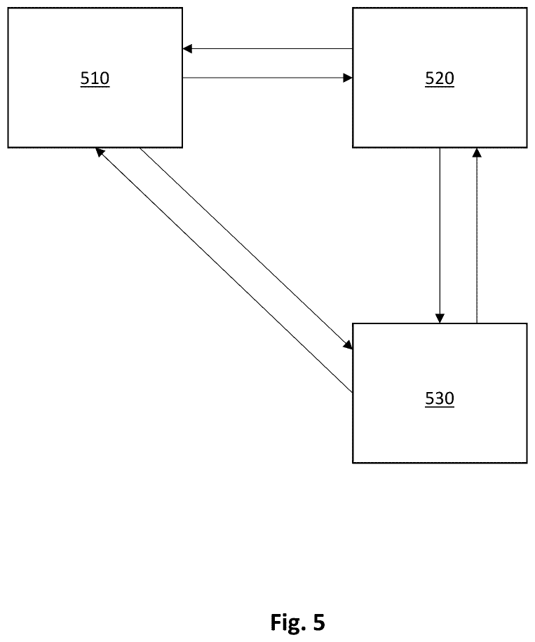

Step 130 of the example method of FIG. 1 includes determining, from the IMU signal, an AVO of a wearer of the wearable device. As with steps 140 and 150 described below, this step may be performed by the wearable device, or may be performed by a connected device (e.g., a client device such as a smartphone or a personal computer, or by a server device) that acquires sensor signals from the wearable device. In particular embodiments, steps 130-150 may be performed by a server device, a client device, the wearable device, or a combination thereof. For example, FIG. 5 illustrates an example system that implements the techniques of FIG. 1. The system of FIG. 5 includes a wearable device 510, a client device 520, and a server device 530, each of which may represent one or more of its respective devices (e.g., server device 530 may include multiple server devices, in particular embodiments). Each device illustrated in the system of FIG. 5 may perform certain steps of FIG. 1, while in particular embodiments only two devices (e.g., a wearable device and either a server device or client device) may be used.

In particular embodiments a wearable device may perform all of the steps of FIG. 1, including the data acquisition steps 110-120 and the processing steps 130-150. In other embodiments, wearable device 510 may perform data acquisition steps 110-120 and may provide the acquired data to client device 520 (which may be, for example, a mobile device such as a smartphone or a computing device such as a PC, etc.), which performs some or all of processing steps 130-150. In particular embodiments, server device 530 may perform some or all of processing steps 130-150 based on data received from wearable device 510 and/or from client device 520. In particular embodiments, a machine-learning model such as the ML model described below may be deployed on one or more of the devices shown in the system of FIG. 5, although a wearable device's typically more limited resources usually means the ML model would be deployed on a client device or on the server device. While the example system of FIG. 5 illustrates bidirectional communication between each component, in particular embodiments communication may occur in only one direction when performing the steps of FIG. 1. (e.g., the wearable device may transmit sensor data to a client device, which the client device may then use to perform certain processing steps without communicating data back to the wearable device).

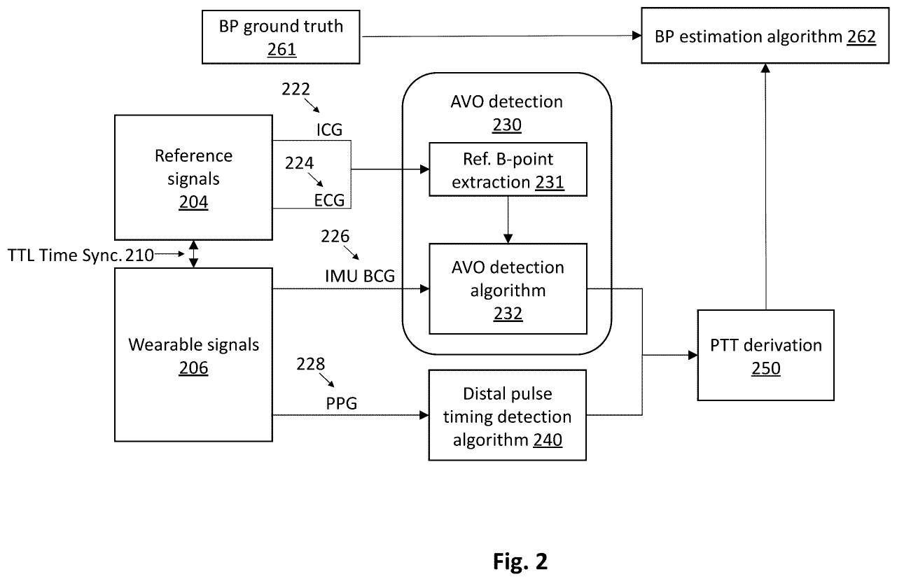

FIG. 2 illustrates an example implementation of an AVO detection block 230, among other things. In the example of FIG. 2, AVO detection block 230 includes AVO detection algorithm 232, which detects the wearer's AVO from BCG signals 226 from the IMU signals of the wearable device.

In the example of FIG. 2, in order to detect a wearer's AVO from the wearable IMU signals, the AVO detection algorithm 230 is first trained from ground-truth reference signals. For example, reference signals 204 may include ECG and ICG signals, which are obtained from several of test subjects. During training, these reference signals 204 are synchronized with acquisition of wearable signals 206 from the wearable device, i.e., with the acquisition of IMU signals 226 and PPG signals 228. For instance, in the example of FIG. 2, TTL (Transistor-Transistor Logic) time synchronization 210 is used to synchronize the reference signals 204 with wearable signals 208. For instance, standard digital pulses (e.g., 0-5V or 03.3V, etc.) may be used to synchronize independent measurement systems. Particular embodiments may route the TTL signal into the MCU of the wearable device (e.g., into an earbud MCU) as an external interrupt, which initiates IMU/PPG sampling simultaneously with acquisition of the external reference signals. For instance, a master device may emit a TTL pulse. The rising edge of this pulse may trigger acquisition of both wearable signals and the reference signals, and those signals may be timestamped relative to the TTL rising edge. In particular embodiments, TTL synchronization may be well under, e.g., 50 μs, as the error margin of TTL signal propagation can be under a few μs and the MCU interrupt handling delay is typically less than 5 μs.

Once synchronized signals are obtained, then ground-truth B-point timing of a test subject's cardiovascular signals can be obtained from the ECG/ICG signals, for example by using any suitable ECG/ICG-based regression model, in reference B-point extraction 231. The ECG (R-wave) and ICG (C-point peak) signals may be aligned, and the B-point may be determined from, e.g., the following regression formula: RB=1.233×RZ−0.0032×RZ²−31.59, where RB represents the time delay between the ECG R-wave and the B-point and RZ represents the time delay between the ECG R-wave and the peak of the dZ/dt function (that peak being the C-point), where dZ/dt is the derivative of the impedance signal. These ground-truth B points can then serve as the ground-truth training data, as described below.

For instance, the AVO detection algorithm 232 can be a machine-learning model (e.g., an autoencoder and a transformer) that is trained to detect B-point signals from BCG signals obtained from the IMU signals of the wearable device, using the ECG/ICG B-point as the ground-truth training labels. Particular embodiments may evaluate multiple BCG fiducial points (H, I J-waves) against the ground truth data; and in particular embodiments, the I-wave may be the best proxy for AVO, due to the I-wave's low mean absolute error. Once trained, then AVO detection algorithm 232 can receive IMU BCG signals and then output predicted B-points from those signals, each of which corresponds to a particular AVO event of the wearer. This disclosure contemplates the AVO detection algorithm may receive IMU BCG signals by receiving data processed from those signals, e.g., by receiving features extracted from the IMU BCG signals, in particular embodiments.

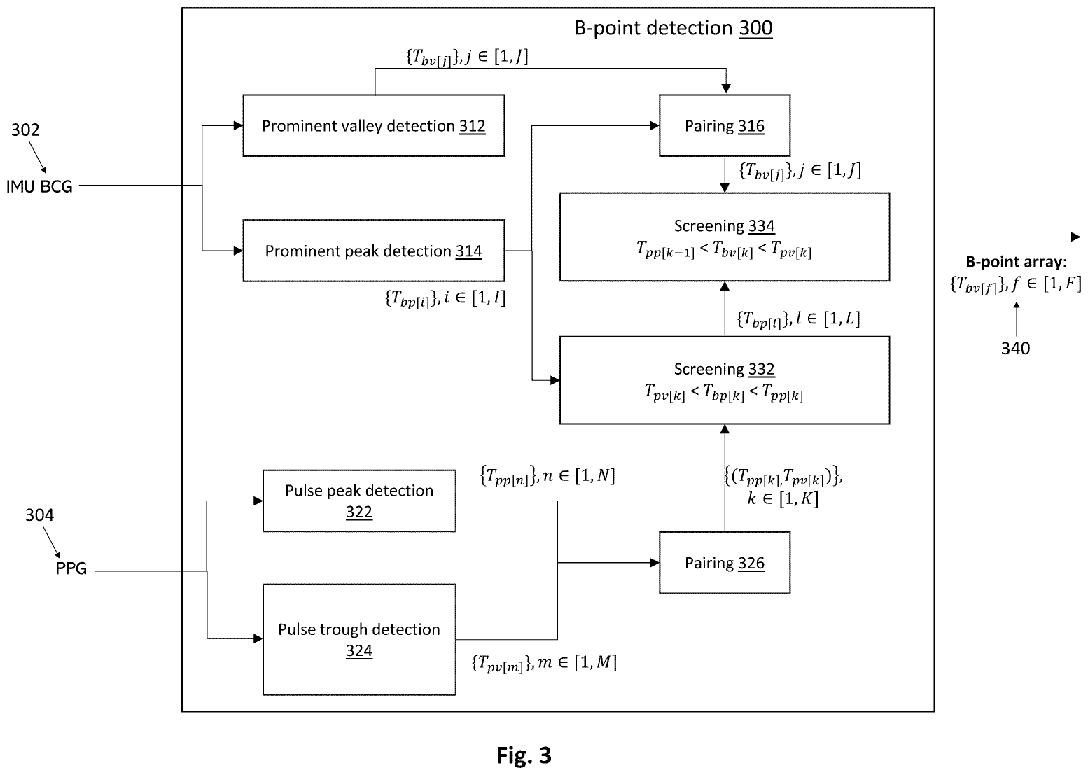

FIG. 3 illustrates an alternative example embodiment of a B-point detection algorithm 300 in which BCG signals from an IMU of the wearable device and aligned PPG signals from the PPG sensor of the wearable device are used together to output a B-point array for AVO detection. In the example of FIG. 3, PPG data 304 are used to detect pulse peaks 322 and pulse troughs 324. Each detection process yields an array; for instance, an array of peaks T_{pp[n]} from the PPG data, where n runs from 1 to N. Likewise, pulse trough detection process 324 yields an array of troughs T_{pv[m]} from the PPG data, where m runs from 1 to M. These two arrays are paired in step 326 by pairing together each peak and trough that are associated with the same heartbeat.

IMU BCG signals 302 (which may be fused signals, as described below) are used to detect prominent valleys 312 and prominent peaks 314. Each detection process yields an array; for instance, an array of valleys T_{bv[j]} from the BCG data, where j runs from 1 to J. Likewise, prominent peak detection process 314 yields an array of peaks T_{bp[i]} from the BCG data, where i runs from 1 to I. These arrays are paired in step 316 by pairing the BCG peaks and valleys that correspond to the same heartbeat.

Screening 332 results in an array of BCG peaks T_{bp[l]} where l runs from 1 to L. This screening occurs by selecting the BCG peaks T_{bp[k]} that are both (1) later than the corresponding PPG troughs T_{pv[k]} and (2) earlier than the corresponding PPG peaks T_{pp[k]}. In other words, the BCG peaks that are between corresponding PPG peaks and PPG troughs are retained; otherwise, the BCG peak is rejected. A second screening step 334 selects an array of BCG valleys T_{bv[k]} that are later than a preceding PPG peak T_{pp[k-1]} and are earlier than a PPG valley T_{pv[k]}. In other words, each related BCG valley that is between a previous PPG peak and the PPG valley from the corresponding heartbeat are retained as a B-point. Here, the BCG valleys that are evaluated in the second screening step are only those that have a paired BCG peak retained from the first screening step, i.e., those for which the BCG peak was not rejected. The resulting output is a B-point array 340 T_{bv[f]} where f runs 1 to F, meaning that there are F total estimated B points in the array after the two screening steps 332 and 334.

In particular embodiments, fusion may be performed on the BCG signals from the IMU sensor in order to improve the signal-to-noise ratio of that modality. For instance, a signal from each axis of an IMU may be used to create axis-specific BCG data. Each axis's BCG signal may be filtered and normalized, e.g., as 0 mean and 1 standard deviation. The normalized BCG signals are then multiplied with each other, in particular embodiments, to form the fused BCG signal. In particular embodiments, normalized BCG signals may be included in the fusion based on their individual signal-to-noise ratios. Thus, for example, if a wearer is leaning over at a 45 angle, the BCG signal quality can be boosted by fusing x-axis and y-axis signals, which carry features related to cardiovascular activities, with a superposition of random and independent noise. In particular embodiments, BCG signal fusion may be performed by an AI model trained to fuse signals based on ground-truth fused signals that have low signal-to-noise.

In particular embodiments, a BCG fusion can be based on a combination of multi-axis accelerometer and gyroscope signals, assigning higher weight to signals that carry more features related cardiovascular activities, which may depend on the wearer's posture.

In particular embodiments, BCG and/or PPG signals from multiple wearable devices may be used to identify AVO events of a wearer. For example, IMU and PPG signals of each (e.g., left and right) of a pair of earbuds may be input to a trained manche-learning model, as described above, which may then output B-points from the input data.

Step 140 of the example method of FIG. 1 includes determining, from the PPG signal, a pulse arrival time of the wearer. For instance, in the example of FIG. 2, a digital pulse extraction algorithm 240 extracts the distal pulse arrival from the synchronized PPG signal to establish the end point for PTT calculation, for example using any suitable extraction technique for such PPG signals. Then, step 150 of the example method of FIG. 1 includes estimating, based on (1) the AVO determined from the IMU signals and (2) the pulse arrival time determined from the PPG signal, a PTT of the wearer, for instance as illustrated by PTT derivation 250 in the example of FIG. 2. Because the AVO signal is precisely obtained and synchronized with the PPG-derived pulse arrival time, the PTT signal can accurately be estimated as the difference between the distal pulse arrival time and the AVO time.

Once the PTT signal is estimated, then it (or, in particular embodiments, the AVO identification itself, without conversion to PTT) can be used to determine a number of cardiovascular metrics. For example, PTT is inversely proportional to blood pressure, and blood pressure may be determined from PTT. For example, PTT may be used along with an arterial wave propagation model to determine arterial elasticity, which along with an arterial wall model can be used to determine blood pressure for an individual. Thus, a person's cardiovascular metrics such as blood pressure can be periodically and non-invasively estimated from a wearable device, including a wearable device (e.g., earbuds) that people already use for a variety of other functions.

In particular embodiments, blood pressure may be estimated from PTT by first gathering ground-truth blood pressure measurements (e.g., by three auscultations in each of the three stages, and/or a finger clamp, etc.), such as BP ground truth 261 in the example of FIG. 2. These ground-truth blood pressure values at particular points in time can be used as training data to train a blood pressure estimation algorithm (e.g, an ML model), such as BP estimation algorithm 262 of FIG. 2, to estimate blood pressure from input PTT data.

In particular embodiments, cardiovascular data may be displayed to a person, such as to a wearer or a health professional. For example, an application (e.g., a health application on a smartphone) may include a UI that provides real-time and/or historical information about a wearer's cardiovascular conditions, such as by displaying blood-pressure determinations, PTT determinations, BCG data, etc. In particular embodiments, notifications may be surfaced to a person (e.g., to the wearer or to a medical professional) if a cardiovascular metric indicates that a dangerous health condition may be occurring or may be imminent.

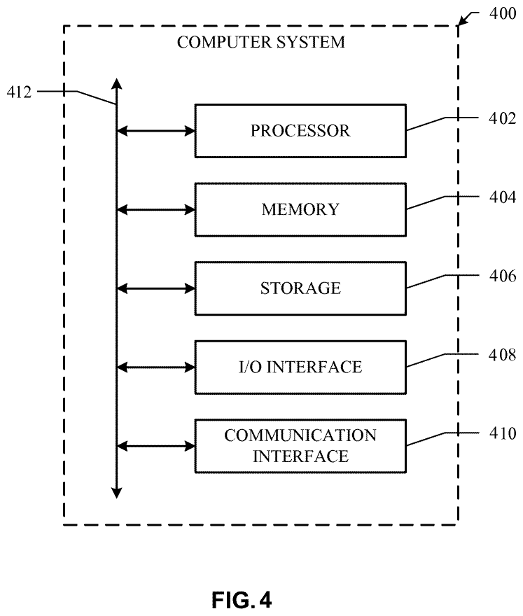

FIG. 4 illustrates an example computer system 400. In particular embodiments, one or more computer systems 400 perform one or more steps of one or more methods described or illustrated herein. In particular embodiments, one or more computer systems 400 provide functionality described or illustrated herein. In particular embodiments, software running on one or more computer systems 400 performs one or more steps of one or more methods described or illustrated herein or provides functionality described or illustrated herein. Particular embodiments include one or more portions of one or more computer systems 400. Herein, reference to a computer system may encompass a computing device, and vice versa, where appropriate. Moreover, reference to a computer system may encompass one or more computer systems, where appropriate.

This disclosure contemplates any suitable number of computer systems 400. This disclosure contemplates computer system 400 taking any suitable physical form. As example and not by way of limitation, computer system 400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 400 may include one or more computer systems 400; be unitary or distributed; span multiple locations; span multiple machines; span multiple data centers; or reside in a cloud, which may include one or more cloud components in one or more networks. Where appropriate, one or more computer systems 400 may perform without substantial spatial or temporal limitation one or more steps of one or more methods described or illustrated herein. As an example and not by way of limitation, one or more computer systems 400 may perform in real time or in batch mode one or more steps of one or more methods described or illustrated herein. One or more computer systems 400 may perform at different times or at different locations one or more steps of one or more methods described or illustrated herein, where appropriate.

In particular embodiments, computer system 400 includes a processor 402, memory 404, storage 406, an input/output (I/O) interface 408, a communication interface 410, and a bus 412. Although this disclosure describes and illustrates a particular computer system having a particular number of particular components in a particular arrangement, this disclosure contemplates any suitable computer system having any suitable number of any suitable components in any suitable arrangement.

In particular embodiments, processor 402 includes hardware for executing instructions, such as those making up a computer program. As an example and not by way of limitation, to execute instructions, processor 402 may retrieve (or fetch) the instructions from an internal register, an internal cache, memory 404, or storage 406; decode and execute them; and then write one or more results to an internal register, an internal cache, memory 404, or storage 406. In particular embodiments, processor 402 may include one or more internal caches for data, instructions, or addresses. This disclosure contemplates processor 402 including any suitable number of any suitable internal caches, where appropriate. As an example and not by way of limitation, processor 402 may include one or more instruction caches, one or more data caches, and one or more translation lookaside buffers (TLBs). Instructions in the instruction caches may be copies of instructions in memory 404 or storage 406, and the instruction caches may speed up retrieval of those instructions by processor 402. Data in the data caches may be copies of data in memory 404 or storage 406 for instructions executing at processor 402 to operate on; the results of previous instructions executed at processor 402 for access by subsequent instructions executing at processor 402 or for writing to memory 404 or storage 406; or other suitable data. The data caches may speed up read or write operations by processor 402. The TLBs may speed up virtual-address translation for processor 402. In particular embodiments, processor 402 may include one or more internal registers for data, instructions, or addresses. This disclosure contemplates processor 402 including any suitable number of any suitable internal registers, where appropriate. Where appropriate, processor 402 may include one or more arithmetic logic units (ALUs); be a multi-core processor; or include one or more processors 402. Although this disclosure describes and illustrates a particular processor, this disclosure contemplates any suitable processor.

In particular embodiments, memory 404 includes main memory for storing instructions for processor 402 to execute or data for processor 402 to operate on. As an example and not by way of limitation, computer system 400 may load instructions from storage 406 or another source (such as, for example, another computer system 400) to memory 404. Processor 402 may then load the instructions from memory 404 to an internal register or internal cache. To execute the instructions, processor 402 may retrieve the instructions from the internal register or internal cache and decode them. During or after execution of the instructions, processor 402 may write one or more results (which may be intermediate or final results) to the internal register or internal cache. Processor 402 may then write one or more of those results to memory 404. In particular embodiments, processor 402 executes only instructions in one or more internal registers or internal caches or in memory 404 (as opposed to storage 406 or elsewhere) and operates only on data in one or more internal registers or internal caches or in memory 404 (as opposed to storage 406 or elsewhere). One or more memory buses (which may each include an address bus and a data bus) may couple processor 402 to memory 404. Bus 412 may include one or more memory buses, as described below. In particular embodiments, one or more memory management units (MMUs) reside between processor 402 and memory 404 and facilitate accesses to memory 404 requested by processor 402. In particular embodiments, memory 404 includes random access memory (RAM). This RAM may be volatile memory, where appropriate Where appropriate, this RAM may be dynamic RAM (DRAM) or static RAM (SRAM) or VRAM. Moreover, where appropriate, this RAM may be single-ported or multi-ported RAM. This disclosure contemplates any suitable RAM. Memory 404 may include one or more memories 404, where appropriate. Although this disclosure describes and illustrates particular memory, this disclosure contemplates any suitable memory.

In particular embodiments, storage 406 includes mass storage for data or instructions. As an example and not by way of limitation, storage 406 may include a hard disk drive (HDD), a floppy disk drive, flash memory, an optical disc, a magneto-optical disc, magnetic tape, or a Universal Serial Bus (USB) drive or a combination of two or more of these. Storage 406 may include removable or non-removable (or fixed) media, where appropriate. Storage 406 may be internal or external to computer system 400, where appropriate. In particular embodiments, storage 406 is non-volatile, solid-state memory. In particular embodiments, storage 406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 406 taking any suitable physical form. Storage 406 may include one or more storage control units facilitating communication between processor 402 and storage 406, where appropriate. Where appropriate, storage 406 may include one or more storages 406. Although this disclosure describes and illustrates particular storage, this disclosure contemplates any suitable storage.

In particular embodiments, I/O interface 408 includes hardware, software, or both, providing one or more interfaces for communication between computer system 400 and one or more I/O devices. Computer system 400 may include one or more of these I/O devices, where appropriate. One or more of these I/O devices may enable communication between a person and computer system 400. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 408 for them. Where appropriate, I/O interface 408 may include one or more device or software drivers enabling processor 402 to drive one or more of these I/O devices. I/O interface 408 may include one or more I/O interfaces 408, where appropriate. Although this disclosure describes and illustrates a particular I/O interface, this disclosure contemplates any suitable I/O interface.

In particular embodiments, communication interface 410 includes hardware, software, or both providing one or more interfaces for communication (such as, for example, packet-based communication) between computer system 400 and one or more other computer systems 400 or one or more networks. As an example and not by way of limitation, communication interface 410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system 400 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 400 may include any suitable communication interface 410 for any of these networks, where appropriate. Communication interface 410 may include one or more communication interfaces 410, where appropriate. Although this disclosure describes and illustrates a particular communication interface, this disclosure contemplates any suitable communication interface.

In particular embodiments, bus 412 includes hardware, software, or both coupling components of computer system 400 to each other. As an example and not by way of limitation, bus 412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 may include one or more buses 412, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
acquiring a motion signal by an inertial measurement unit (IMU) sensor of a wearable device;
acquiring a photoplethysmogram (PPG) signal by a PPG sensor of the wearable device, wherein the PPG sensor is synchronized with the IMU sensor;
determining, from the IMU signal, an aortic valve opening (AVO) of a wearer of the wearable device;
determining from the PPG signal, a pulse arrival time of the wearer; and
estimating, based on (1) the AVO determined from the IMU signals and (2) the pulse arrival time determined from the PPG signal, a pulse transit time (PTT) of the wearer.

2. The method of claim 1, wherein the wearable device comprises an earbud.

3. The method of claim 2, wherein the PPG sensor is positioned in the earbud such that the PPG sensor is at a concha of the wearer.

4. The method of claim 1, wherein the wearable device further comprises one or more of a microphone or a body-temperature sensor.

5. The method of claim 1, wherein acquiring the motion signal and acquiring the PPG signal comprise simultaneously triggering, by a master clock of a controller unit of the wearable device, acquisition of the motion signal by the IMU sensor and acquisition of the PPG signal by the PPG sensor.

6. The method of claim 1, further comprising:
operating the wearable device in a first sensing mode comprising acquiring signals from an accelerometer of the IMU at a first sampling rate;
determining, from the acquired accelerometer signals, whether a user is stationary;
in response to a determination that the user is not stationary, then continuing to operate the wearable device in the first sensing mode; and
in response to at least a determination that the user is stationary, then operating the wearable device in a second, PTT sensing mode comprising the steps of claim 1, wherein in the second, PTT sensing mode, the IMU sensor and the PPG sensor acquire respective signals at a second sampling rate that is greater than the first sampling rate.

7. The method of claim 6, wherein in response to at least a determination that the user is stationary, then operating the wearable device in a second, PTT sensing mode comprises operating the wearable device in the second, PTT sensing mode in response to a determination that the weather is stationary and one or more of (1) an elapsed time since the wearable device previously operated in the second, PTT sensing mode or (2) a cardiovascular risk profile of the wearer.

8. The method of claim 1, wherein determining, from the IMU signal, the AVO of the wearer comprises:
providing, to a trained machine-learning (ML) model, the IMU signal;
receiving, from the trained ML model, a plurality of estimated B points of the wearer's heartbeats; and
determining, from the plurality of estimated B points of the wearer's heartbeats, a corresponding plurality of AVOs of the wearer.

9. The method of claim 1, wherein determining, from the IMU signal, the AVO of the wearer comprises:
determining, from the IMU signal and from a synchronized PPG signal, an array of estimated B points of the wearer's heartbeats; and
determining, from the array of estimated B points, corresponding AVOs of the wearer.

10. The method of claim 1, further comprising:
fusing a plurality of BCG signals from the motion signal of the IMU, each BCG signal associated with a particular IMU axis; and
determining, from the fused BCG signal, the AVO of the wearer.

11. The method of claim 1, further comprising estimating, from the estimated PTT of the wearer, a corresponding blood pressure of the wearer.

12. The method of claim 1, further comprising providing, for presentation on a user interface displayed on an electronic device, information associated with the estimated PTT of the wearer.

13. A system comprising:
a wearable device comprising:
an inertial measurement unit (IMU) sensor configured to acquire a motion signal of a wearer;
a photoplethysmogram (PPG) sensor configured to acquire a PPG signal of the wearer, wherein the PPG sensor is synchronized with the IMU sensor; and
one or more non-transitory computer readable storage media storing instructions; and one or more processors coupled to the one or more non-transitory computer readable storage media and operable to execute the instructions to:
determine, from the IMU signal, an aortic valve opening (AVO) of a wearer of the wearable device;
determine from the PPG signal, a pulse arrival time of the wearer; and
estimate, based on (1) the AVO determined from the IMU signals and (2) the pulse arrival time determined from the PPG signal, a pulse transit time (PTT) of the wearer.

14. The system of claim 13, wherein the wearable device comprises an earbud.

15. The system of claim 13, wherein the wearable device further comprises a controller unit comprising a master clock, the controller unit configured to simultaneously trigger acquisition of the motion signal by the IMU sensor and acquisition of the PPG signal by the PPG sensor.

16. The system of claim 13, wherein the wearable device is configured to:
operate in a first sensing mode comprising acquiring signals from an accelerometer of the IMU at a first sampling rate;
determine, from the acquired accelerometer signals, whether a user is stationary;
in response to a determination that the user is not stationary, then continue to operate the wearable device in the first sensing mode; and
in response to at least a determination that the user is stationary, then operate the wearable device in a second, PTT sensing mode, wherein in the second, PTT sensing mode, the IMU sensor and the PPG sensor acquire respective signals at a second sampling rate that is greater than the first sampling rate.

17. The system of claim 13, wherein determining, from the IMU signal, the AVO of the wearer comprises:
providing, to a trained machine-learning (ML) model, the IMU signal;
receiving, from the trained ML model, a plurality of estimated B points of the wearer's heartbeats; and
determining, from the plurality of estimated B points of the wearer's heartbeats, a corresponding plurality of AVOs of the wearer.

18. The system of claim 13, further comprising one or more processors that are operable to execute the instruction to estimate, from the estimated PTT of the wearer, a corresponding blood pressure of the wearer.

19. The system of claim 13, wherein the wearable device comprises the one or more non-transitory computer readable storage media and the one or more processors.

20. One or more non-transitory computer-readable storage media storing instructions that are operable when executed by one or more processors to:
acquire a motion signal by an inertial measurement unit (IMU) sensor of a wearable device;
acquire a photoplethysmogram (PPG) signal by a PPG sensor of the wearable device, wherein the PPG sensor is synchronized with the IMU sensor;
determine, from the IMU signal, an aortic valve opening (AVO) of a wearer of the wearable device;
determine from the PPG signal, a pulse arrival time of the wearer; and
estimate, based on (1) the AVO determined from the IMU signals and (2) the pulse arrival time determined from the PPG signal, a pulse transit time (PTT) of the wearer.
